(19) United States
(12) Patent Application Publication
(10) Pub. No.: US 2023/0031262 A1
(43) Pub. Date: Feb. 2, 2023

(54) TREATMENT OF MILD TRAUMATIC BRAIN INJURY

(71) Applicant: OXEIA BIOPHARMACEUTICALS, INC., San Diego, CA (US)

(72) Inventors: Vishal BANSAL, San Diego, CA (US); Kartik SHAH, San Diego, CA (US); Michael WYAND, San Diego, CA (US)

(21) Appl. No.: 17/787,848

(22) PCT Filed: Dec. 22, 2020
(86) PCT No.: PCT/US2020/066703

§ 371 Date: Jun. 21, 2022

Related U.S. Application Data
(60) Provisional application No. 62/953,130, filed on Dec. 23, 2019.

(51) Int. Cl. A61K 38/22; A61K 9/00; A61K 9/70; A61P 25/00

(52) U.S. Cl. CPC A61K 38/22; A61K 9/0019; A61K 9/7023; A61P 25/00

(57) ABSTRACT

Disclosed are methods for mitigating one or more debilitating symptoms of a mTBI for a patient diagnosed with sustained mTBI. These methods comprise administering an effective amount of ghrelin or a variant thereof over multiple consecutive days after the diagnosis of a sustained mTBI.

### FIELD

This disclosure is directed to methods for treating sustained mild traumatic brain injuries which injuries include, by way of example, concussions, and other such neurological disorders. The methods employ an effective amount of a composition comprising ghrelin or a ghrelin variant. This disclosure is also directed to a kit of parts comprising multiple doses of ghrelin or a variant thereof.

### STATE OF THE ART

Mild traumatic brain injuries (mTBI), typically including concussions, having “your bell rung,” and the like, describe an insult to the brain that, in turn, can cause long term injury to the brain. It most often occurs from direct contact to the head, but can also result from indirect injury (e.g., whiplash injury or violent shaking of the head). Individuals who have suffered one brain injury are more at risk for a second brain injury and more susceptible for subsequent injuries. The damage from successive mTBIs is cumulative. (Cantu, R.C., Second-impact syndrome, Clinics in Sports Medicine, 17(I):37-44, 1998).

The long term damage arising from mTBI include cognitive and motor skill deterioration such as psychomotor slowing, poor concentration and attention retrieval resulting in increased variability of performance, and overall executive dysfunction, as well as sleep dysfunction, and emotional/behavioral changes (Stuns, et al., ‘Adult Clinical Neuropsychology: Lessons from Studies of the Frontal Lobes’, Annual Review of Psychology, 53, 401-433 (2003)). Common examples of long term effects of mTBI are found in soldiers, boxers, soccer players, and the like. There are well-documented examples of individuals who, long after the occurrence of the mTBI(s), begin to manifest the cumulative damage to the brain by loss of one or more cognitive skills and/or motor skills.

The difference between mTBI and other brain disorders is that mTBI is caused by one or more injuries as opposed to an underlying disease modality. That is, the injuries to the brain cannot be attributed to an underlying pathology but, rather are the results of the injuries.

Short-term symptoms of mTBI include, among others, headaches, loss of clarity or confusion, difficulty in focusing, double vision, blurry vision, sleep dysfunction, emotional/behavioral changes, emotional outbursts, and loss of memory. Described herein are improvements in treating mTBI, including mTBI that is sustained or poorly resolved.

### SUMMARY

Many of the symptoms of mTBI are debilitating and most resolve themselves with a few days up to a week after the injury occurred. However, in some cases, some or all of the symptons are not adequately resolved. As described herein, patients who have not resolved one or more of these debilitating symptoms within a week of the occurrence of an mTBI can be classified as experiencing a sustained mTBI. Such is readily distinguishable from post concussion syndrome (PCS). PCS relates to a set of symptoms that continue for weeks, months or even a year after the injury. In the case of a sustained mTBI, the symptoms have not extended so long as to characterized the patient as having a PCS. Rather, the patient with a sustained mTBI wishes to facilitate resolution of the symptoms of that mTBI as soon as practical.

Treatment of mTBI with ghrelin or a variant thereof has been described, for example, in U.S. Pat. Application Publication No. 2017/0281732 which is incorporated herein by reference in its entirety. For example, ghrelin can be administered within 72 hours after the occurrence of the injury.

Surprisingly, it has now been determined that ghrelin treatment mitigates one or more symptoms of mTBI in a subject with a sustained mTBI, even several days to weeks after the initial injury. This disclosure is directed in part to methods for mitigating one or more debilitating symptoms of a mTBI for a patient diagnosed with a sustained mTBI. In particular, this disclosure involves the administration of ghrelin or a variant thereof over one or multiple consecutive days after the diagnosis of a sustained mTBI.

The present disclosure also relates to treatment of mTBI with multiple doses of ghrelin or variant thereof. That is, in one embodiment, mTBI (acute, sustained, and/or PCS) may be treated by administering ghrelin or a variant thereof two or more times a day for at least one day. In one embodiment, mTBI (acute, sustained, and/or PCS) may be treated by administering ghrelin or a variant thereof one or more times per day for at least 2 days.

In one embodiment, ghrelin administration is continued until the patient’s symptoms are resolved. In one embodiment, ghrelin administration is continued until the patient is able to resume normal activities. In one embodiment, ghrelin administration is continued until the patient is cleared by a clinician to resume normal activities. “Normal activities” may be any activities that were interrupted by the mTBI. For example, the patient may have been instructed (e.g., by a clinician) not to perform certain tasks, not to return to work, school, sport activities, or some other activity due to the mTBI. Alternatively, the patient may have been unable to perform the task, and/or the task may have been uncomfortable or otherwise undesireable, due to the mTBI.

In one embodiment, there is provided a method for mitigating one or more debilitating symptoms of mTBI for a patient diagnosed with a sustained mTBI, which method includes administering to the patient an effective amount of ghrelin or a variant thereof over multiple consecutive days after the diagnosis of a sustained mTBI. The methods can include selection or identification of a patient exhibiting one or more symptoms of a sustained mTBI.

In one embodiment, administration of ghrelin or a variant takes the form of a continuous release patch or transdermal device which is optionally replaced during treatment. In one embodiment, ghrelin or a variant is administered orally, e.g., sublingually. In one embodiment, ghrelin or a variant is administered by injection.

In one embodiment, ghrelin administration takes the form of one or more administration(s). For example, in some embodiments, the administration can be one or more administrations for 1-2 days. In some embodiments, the administration can be one or more administrations per day for a period of at least 3 days and preferably at least 5 days and more preferably at least 7 days after diagnosis of a sustained mTBI. In an embodiment, only a single administration per day is employed and administration is done on a daily basis. In another embodiment, administration takes the form of a medicament loaded syringe.

In one embodiment, there is a provided a kit of parts comprising a plurality of single dose compositions of ghrelin each composition marked, labeled or otherwise identified for administration on a treatment schedule. In one embodiment, the concentration for each single dose composition of ghrelin is tapered from an initial dose to a final dose over a period of at least 3 days, preferably at least 5 days and more preferably at least 7 days. In an embodiment, the concentration of ghrelin is reduced from the first dose to the final dose such that the last dose has a ghrelin concentration that is no more than 50% of the initial dose.

### DETAILED DESCRIPTION

Disclosed are methods for treating sustained mild brain injuries as well as a kit of parts. However, prior to providing further details, the following terms will be defined. If not defined, terms used herein have their generally accepted scientific meaning.

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by ( + ) or ( - ) 10%, 5%, 1%, or any subrange or subvalue there between. Preferably, the term “about” when used with regard to a dose amount means that the dose may vary by +/-10%.

“Comprising” or “comprises” is intended to mean that the compositions and methods include the recited elements, but not excluding others.

“Consisting essentially of” when used to define compositions and methods, shall mean excluding other elements of any essential significance to the comTBInation for the stated purpose. Thus, a composition consisting essentially of the elements as defined herein would not exclude other materials or steps that do not materially affect the basic and novel characteristic(s) of the claimed disclosure.

“Consisting of” shall mean excluding more than trace elements of other ingredients and substantial method steps. Embodiments defined by each of these transition terms are within the scope of this disclosure.

The term “ghrelin” refers to the naturally occurring peptide comprising 28 amino acids that include an N-octanoyl group at the 3-position of serine. The amino acid sequence of ghrelin is known.

The term “ghrelin variant” refers to peptides that include a C-terminal alkyl ester (—COOR) where R is C₁-C₆ alkyl, C₂-C₆ alkenyl or C₂-C₆ alkynyl; a N-terminal amide (R¹C(O)NH—) where R¹ is C₁-C₆ alkyl, C₂-C₆ alkenyl or C₂-C₆ alkynyl; a comTBInation of a C-terminal alkyl ester and a N-terminal amide both as defined above; and/or a ghrelin variant as described in U.S. Pat. Application Publication No. 2017/0281732, which is incorporated herein by reference in its entirety, including for all compositions, formulations, and methods taught therein.

The term “administration” refers to dosing a patient with an effective amount of a composition comprising ghrelin (or variant) wherein the dosing can be a single administration, continuous or intermittent or by several subdoses that in the aggregate provide for a single dose. For example, dosing may be continued throughout the course of treatment that may be as short as 1-2 days, 3 days or as long as 7 or more days such as 10 days, 12 days or 14 days, wherein treatment is initiated after diagnosis of a sustained mTBI. The route of administration is selected by the attending clinician and is based on factors such as the age, weight and general health of the patient as well as the severity of the condition. Suitable routes include parenteral, intravenous, transdermal, vaginal, nasal, sublingual, pulmonary, and the like.

The term “asymptomatic” means that the patient reports that s/he has no remaining debilitating symptoms of the sustained mTBI. While some mild symptoms may persist, debilitating symptoms such as, by way of example only and without limitation, debilitating headaches, double vision, blurred vision, nausea, migranes, and confusion have abated. In some cases, the patients is able to resume most if not all of his/her day-to-day or normal activities.

The term “debilitating” as it relates to a mTBI means that one or more symptoms of that mTBI are such that the patient is unable to function in his or her accustomed manner.

The term “mild traumatic brain Clinically, traumatic brain injury can be rated as mild, moderate or severe based on TBI variables that include duration of loss of consciousness (LOC), Glasgow Coma Score (GCS) and post traumatic stress amnesia (see, e.g., Levin et al., “The Galveston Orientation and Amnesia Test: a practical scale to assess cognition after head injury,” J Nervous Mental Dis 167: 675-84 (1979); Holm et al.,“ J. Neurotrauma task force on mild traumatic brain injury of the WHO Collaborating Centre. Summary of the WHO Collaborating Centre for Neurotrauma Task Force on Mild Traumatic Brain Injury,” J Rehabil Med 37:137-41 (2005)). For example, a brain injury can be classified as a mild brain injury when a patient has a GCS score of 13-15, post-traumatic amnesia of less than 1 day, and/or a LOC of between 0 to 30 minutes.

The term “sustained mTBI” (sometimes referred to as a “poorly resolved mTBI”) means that one or more symptoms of a mTBI are not resolved after several days to weeks after the initial injury such that the patient remains debilitated. In one embodiment, one or more of the symptoms of the mTBI persist for at least 7 days after the injury. However, a sustained mTBI is not to be confused with a PCS which requires a much prolonged duration of symptoms (typically about 30 days or more) than that of a sustained mTBI.

### Methods

The methods described herein treat patients who are diagnosed as having a sustained mTBI. Such a diagnosis and selection or identification of such patients may be made based on the continuation of debilitating symptoms for mTBI, for example at least 3, 4, 5, 6, or 7 days after the insult arising from the mTBI, without a diagnosis of PCS. Many patients who suffer from mTBI do not seek immediate medical treatment and only do so when the debilitating symptoms of that mTBI do not resolve in a timely manner.

Unlike patients treated immediately after the mTBI, patients suffering from a sustained mTBI have allowed the biological cascade of adverse events in the brain to continue unabated. Such events include untreated inflammation in the brain due, for example, to metabolic derangement, neuronal damage, or inflammation associated with overproduction of reactive oxygen species (ROS). This disclosure addresses the need to both mitigate the patient’s debilitating symptoms while addressing these unabated adverse events.

The methods described herein further relate to treatment of patients having mTBI with ghrelin or variant thereof by administering multiple doses (administrations) and/or for multiple days. In embodiments, the mTBI is acute mTBI. In embodiments, the mTBI is sustained mTBI. In embodiments, the mTBI is PCS. In embodiments, the mTBI is not acute mTBI. In embodiments, the mTBI is not PCS.

In the methods described herein, ghrelin or a variant thereof is administered as a one day treatment, and/or daily ghrelin administration for multiple consecutive days after diagnosis.

For sustained mTBI, such treatment may be initiated at least 3 days after the mTBI. In embodiments, treatment with ghrelin or variant is initiated at least 4 days after the mTBI. In embodiments, treatment with ghrelin or variant is initiated at least 5 days after the mTBI. In embodiments, treatment with ghrelin or variant is initiated at least 6 days after the mTBI. In embodiments, treatment with ghrelin or variant is initiated at least 7 days after the mTBI. In embodiments, treatment with ghrelin or variant is initiated at least 8 days after the mTBI. In embodiments, treatment with ghrelin or variant is initiated at least 9 days after the mTBI. In embodiments, treatment with ghrelin or variant is initiated at least 10 days after the mTBI. In embodiments, treatment with ghrelin or variant is initiated at least 2 weeks after the mTBI. In embodiments, treatment with ghrelin or variant is initiated at least 4 weeks after the mTBI. In embodiments, treatment with ghrelin or variant is initiated between about 3 days and about 30 days after the mTBI. In embodiments, treatment with ghrelin or variant is initiated between more than 3 days and about 30 days after the mTBI. In embodiments, treatment with ghrelin or variant is initiated between about 4 days and about 30 days after the mTBI. In embodiments, treatment with ghrelin or variant is initiated between about 7 days and about 30 days after the mTBI. In embodiments, treatment with ghrelin or variant is initiated between about 7 days and about 28 days after the mTBI. In embodiments, treatment with ghrelin or variant is initiated between about 7 days and about 21 days after the mTBI. The number of days may be any value or subrange within the recited ranges, including endpoints.

In embodiments, ghrelin or a composition comprising ghrelin is administered. In embodiments, a ghrelin variant or a composition comprising a ghrelin variant is administered.

Ghrelin or a variant can be administered in single adminstration or multiple administrations for a day or for multiple days. In one embodiment, the ghrelin or variant can be administered each day for at least 1 day. In one embodiment, the ghrelin or variant can be administered each day for at least 2 days.In one embodiment, the ghrelin or variant can be administered each day for at least 3 days. In one embodiment, the ghrelin or variant can be administered each day for at least 4 days. In one embodiment, the ghrelin or variant can be administered each day for at least 5 days. In one embodiment, the ghrelin or variant can be administered each day for at least 6 days. In one embodiment, the ghrelin or variant can be administered each day for at least 7 days. In one embodiment, the ghrelin or variant can be administered each day for at least 8 days. In one embodiment, the ghrelin or variant can be administered each day for at least 9 days. In one embodiment, the ghrelin or variant can be administered each day for at least 10 days. In one embodiment, the ghrelin or variant can be administered each day for at least 11 days. In one embodiment, the ghrelin or variant can be administered each day for at least 12 days. In one embodiment, the ghrelin or variant can be administered each day for at least 13 days. In one embodiment, the ghrelin or variant can be administered each day for at least 14 days. In some cases, daily ghrelin or variant administration is continued up to 14 days. In an embodiment, ghrelin or variant can be administered daily for between 1 and 14 days. In an embodiment, ghrelin or variant can be administered for more than 14 days. In an embodiment, ghrelin or variant can be administered until one or more symptoms of the mTBI (or sustained mTBI or PCS) is resolved. In an embodiment, the ghrelin or variant can be administered continuously (e.g., using a transdermal patch) for between 1 and 14 days or more.

In one embodiment, ghrelin or variant can be administered at least once per day. In one embodiment, ghrelin or variant can be administered at least twice per day. In one embodiment, ghrelin or variant can be administered at least 3 times per day. In one embodiment, ghrelin or variant can be administered at least 4 times per day. In one embodiment, ghrelin or variant can be administered at least 5 times per day. In one embodiment, ghrelin or variant can be administered once per day. In one embodiment, ghrelin or variant can be administered twice per day. In one embodiment, ghrelin or variant can be administered 3 times per day. In one embodiment, ghrelin or variant can be administered 4 times per day. In one embodiment, ghrelin or variant can be administered 5 times per day.

Currently, patients with acute mTBI are sent home once a clinician has determined that the mTBI does not pose an immediate risk. In contrast, patients having sustained mTBI, e.g., unresolved symptoms that last days, weeks, or months after the intial injury, are treated based on symptoms presented. In an embodiment is provided a treatment for sustained mTBI, e.g. treatment that is significantly later in time than the acute injury and where the underlying adverse events in the brain have gone on unabated. These methods may be predicated on a dosing schedule that requires daily ghrelin administration over the course of one or several days so as to both mitigate the debilitating symptoms of the mTBI and to address the adverse condition of the brain.

In one embodiment, a patient having sustained mTBI is selected for treatment. In one embodiment, a patient having one or more symptoms (e.g., debilitating symptoms) of mTBI at least 3 days after injury is selected. In one embodiment, a patient having one or more symptoms (e.g., debilitating symptoms) of mTBI at least 4 days after injury is selected. In one embodiment, a patient having one or more symptoms (e.g., debilitating symptoms) of mTBI at least 5 days after injury is selected. In one embodiment, a patient having one or more symptoms (e.g., debilitating symptoms) of mTBI at least 6 days after injury is selected. In one embodiment, a patient having one or more symptoms (e.g., debilitating symptoms) of mTBI at least 7 days after injury is selected.

In embodiments, a patient having acute mTBI is selected. In embodiments, a patient who does not have acute mTBI (e.g., at least 3, at least 7, e.g. days after injury) is selected. In embodiments, a patient having PCS is selected. In embodiments, a patient who has not been diagnosed as having PCS is selected.

In one embodiment, there is a provided a kit of parts comprising a plurality of single dose compositions of ghrelin each composition marked, labeled or otherwise identified for administration on a treatment schedule. In one embodiment, the concentration for each single dose composition of ghrelin is the same throughout the treatment period. In another embodiment, the concentration of ghrelin is reduced or tapered from an initial first dose to a final dose over a period of at least 1, 2, or 3 days, preferably at least 5 days and more preferably at least 7 days. In an embodiment, the concentration of ghrelin is reduced from the first dose to the last dose such that the last dose has a ghrelin concentration that is no more than about 50% of the first dose.

In one embodiment, the ghrelin concentration changes at least twice during the treatment period. Example ghrelin concentration changes are provided in Table 1.

Table 1

| Example | Ghrelin concentration at each day after the start of treatment | | | | | | |
|---|---|---|---|---|---|---|---|
| | 1 | 2 | 3 | 4 | 5 | 6 | 7 |
| A | 100% | 100% | 100% | 100% | 100% | 100% | 100% |
| B | 100% | 100% | 100% | | | | |
| C | 100% | 90% | 80% | 70% | 60% | 50% | 50% |
| D | 100% | 80% | 60% | 50% | 50% | | |
| E | 100% | 90% | 90% | 80% | | | |
| F | 100% | 80% | 60% | | | | |

In one embodiment, mTBI is diagnosed using one or more diagnostic devices or protocols. In one embodiment, the methods provided herein are used in conjunction with one or more recovery protocols. For example, a potential brain injury can be diagnosed and/or monitored utilizing the BTrackS™ System (balancetrackingsystems.com/; Balance Tracking Systems Inc.), utilizing the NFL Concussion Tool, “sports concussion assessment tool” (“SCAT-2;” static.nfl.com/static/content/public/photo/2014/02/20/0ap2000000327062.pdf, which is incorporated herein by reference in its entirety) or other similar tools utilized by the NHL, the NBA, FIFA, Rugby leagues and unions, boxing organizations, etc. Examples include, SCAT-3, ImPACT,ICD-10, nPITEST, acute concussion evaluation (“ACE”), King-Devick, and the like. Other diagnostics or assessments can utilize serum biomarkers (Glial Fibrilliary Acid Protein (GFAP); see for example, Mannix et al., “Serum Biomarkers Predict Acute Symptom Burden in Children after Concussion: A Preliminary Study,” J Neurotrauma 31:1072-1075 (Jun. 1, 2014), which is incorporated herein by reference in its entirety), radiology imaging, self-reporting, accelerometers (for example, in helmets), and the like.

In one embodiment, a subject administered ghrelin or variant as described herein may show improvement on one or more assessment tools, when evaluated before, during, and/or after the administration. Any suitable tool may be used, as would be recognized by one of skill in the art. In one embodiment, the subject shows improvement in number of symptoms and/or severity in sub-acute concussion. In one embodiment, the subject shows improvement on the Post-Concussion Symptom Score questionnaire (PCSS) (available at: hawaiiconcussion.com/downloads/Post-Concussion-Symptom-Scale.pdf, which is incorporated by reference herein in its entirety). The PCSS may be used as an overall measure of symptom burden. The number of symptoms and severity at any time before, during, and/or after treatment may be measures of interest.

In one embodiment, the subject shows improvement in quality of life. In one embodiment, the subject shows improvement on the Quality of Life after Brain Injury scale (QOLIBRI) (Qolibri. Quality of Life Assessment for TBI. Available from: qolibrinet.com, which is incorporated herein by reference in its entirety). The QOLIBRI is a 37 item instrument specifically developed to assess health-related quality of life (HRQoL) of individuals after traumatic brain injury.

In one embodiment, the subject shows improvement on the Patient Global Assessment of Status (PGAS). This tool may utilize a visual analog scale (VAS) to simply assess the patient’s global assessment of their symptoms via the question “How would you rate the effect of your symptoms on how you feel or function today?”

In one embodiment, the subject shows improved cognitive function. In one embodiment, the subject shows improvement on BrainCheck. See, Yang, S., et al., Diagnostic accuracy of tablet-based software for the detection of concussion. PLoS One, 2017. 12(7): p. e0179352, which is incorporated herein by reference in its entirety. In one embodiment, the subject shows improvement in one or more of the tests measured by BrainCheck. BrainCheck is a validated digital assessment tool to aid in the diagnosis of mild cognitive decline. The tool measures a battery of 7 tests to measure cognition, reaction time, and balance. BrainCheck is a Class II medical device by the FDA. BrainCheck assessments include a coordination balance test measuring static and dynamic balance using the Ebbinghaus Illusion, a digit symbol substitution test for general cognitive performance, the Flanker test measuring visual attention, the Stroop Effect measuring reaction time, Trails A&B measuring visual attention and task switching and Recall tests to measure immediate and delayed memory. All scoring algorithms compare test results to a normative age matched dataset.

In one embodiment, the subject shows improvement in any one or more of the assessments of more than about 10%. In one embodiment, the subject shows improvement in any one or more of the assessments of more than about 15%. In one embodiment, the subject shows improvement in any one or more of the assessments of more than about 20%. In one embodiment, the subject shows improvement in any one or more of the assessments of more than about 25%. In one embodiment, the subject shows improvement in any one or more of the assessments of more than about 30%. In one embodiment, the subject shows improvement in any one or more of the assessments of more than about 35%. In one embodiment, the subject shows improvement in any one or more of the assessments of more than about 40%. In one embodiment, the subject shows improvement in any one or more of the assessments of more than about 45%. In one embodiment, the subject shows improvement in any one or more of the assessments of more than about 50%. In one embodiment, the subject shows improvement in any one or more of the assessments of more than about 60%. In one embodiment, the subject shows improvement in any one or more of the assessments of more than about 70%. In one embodiment, the subject shows improvement in any one or more of the assessments of more than about 80%. In one embodiment, the subject shows improvement in any one or more of the assessments of more than about 90%. In one embodiment, the subject shows improvement in any one or more of the assessments of up to about 100%. The improvement may be between any two time points, e.g., before, during, and/or after administration of ghrelin or variant.

### Pharmaceutical Compositions

Ghrelin or a variant thereof will be administered in a therapeutically effective amount by any of the accepted modes of administration suitable for delivery of a peptide. The actual amount of ghrelin or a variant thereof is dependent upon numerous factors such as the severity of the symptoms to be treated, the age and otherwise relative health of the subject, the route and form of administration, and other factors well-known to the skilled artisan.

An effective amount or a therapeutically effective amount or dose of ghrelin or a variant thereof refers to that amount that results in amelioration of debilitating symptoms in a patient. Specific dosages may vary within a range depending upon the dosage form employed and/or the route of administration utilized. The exact formulation, route of administration, dosage, and dosage interval should be chosen according to methods known in the art, in view of the specifics of a subject’s condition.

In one embodiment, the effective amount of ghrelin or a variant thereof ranges from about 10 ng/kg to about 10 mg/kg per day. In one embodiment, the effective amount of ghrelin or a variant thereof ranges from about 1 µg/kg to about 10 mg/kg per day. In one embodiment, the effective amount of ghrelin or a variant thereof ranges from about 1 µg/kg to about 1 mg/kg per day. In one embodiment, the effective amount of ghrelin or a variant thereof ranges from about 10 µg/kg to about 1 mg/kg per day. In one embodiment, the effective amount of ghrelin or a variant thereof ranges from about 20 µg/kg to about 1 mg/kg per day. In one embodiment, the effective amount of ghrelin or a variant thereof ranges from about 30 µg/kg to about 1 mg/kg per day. In one embodiment, the effective amount of ghrelin or a variant thereof ranges from about 40 µg/kg to about 1 mg/kg per day. In one embodiment, the effective amount of ghrelin or a variant thereof ranges from about 50 µg/kg to about 1 mg/kg per day. In one embodiment, the effective amount of ghrelin or a variant thereof ranges from about 60 µg/kg to about 1 mg/kg per day. In one embodiment, the effective amount of ghrelin or a variant thereof ranges from about 70 µg/kg to about 1 mg/kg per day. In one embodiment, the effective amount of ghrelin or a variant thereof ranges from about 80 µg/kg to about 1 mg/kg per day. In one embodiment, the effective amount of ghrelin or a variant thereof ranges from about 90 µg/kg to about 1 mg/kg per day. In one embodiment, the effective amount of ghrelin or a variant thereof ranges from about 100 µg/kg to about 1 mg/kg per day. In one embodiment, the effective amount of ghrelin or a variant thereof ranges from about 10 µg/kg to about 0.1 mg/kg per day. The effective amount may be any value or subrange within the recited ranges, including endpoints.

This invention is not limited to any particular composition or pharmaceutical carrier, as such may vary. In general, ghrelin or a variant thereof will be administered as pharmaceutical compositions by any one of the following routes: oral, systemic (e.g., transdermal, intranasal or by suppository), or parenteral (e.g., intramuscular, intravenous or subcutaneous) administration. In embodiments, administration is parenteral using a dosage regimen that can be adjusted as provided above. Other pharmaceutical compositions can take the form of tablets, pills, capsules, semisolids, powders, sustained release formulations, solutions, suspensions, elixirs, aerosols, or any other appropriate compositions.

Pharmaceutical dosage forms of a compound of this invention may be manufactured by any of the methods well-known in the art, such as, for example, by conventional mixing, sieving, dissolving, melting, granulating, dragee-making, tableting, suspending, extruding, spray-drying, levigating, emulsifying, (nano-/micro-) encapsulating, entrapping, or lyophilization processes. As noted above, the compositions of this invention can include one or more physiologically acceptable inactive ingredients that facilitate processing of active molecules into preparations for pharmaceutical use.

As noted previously, one pharmaceutical composition for use in the methods described herein is a sterile, aqueous composition such as those suitable for intravenous or intramuscular injection. In one embodiment, such compositions are preloaded into syringes for use by the clinician or the patient. Preferably, the syringes are loaded into a container and are labeled, marked or otherwise identified as for use on a given day. For example, in a 7 day treatment regimen, the identication for each syringe will indicate that it is for day 1, or day 2 and so on.

Alternatively, the pharmaceutical composition can take the form of a transdermal patch that provides for continuous release of ghrelin or a variant thereof in amounts such that the aggregate delivered in a given day is an effective amount as provided above. Given that ghrelin has a serum half-life of about 30 minutes, continuous release allows for continuous presence in the serum as well as in the brain.

When administration of ghrelin is via a transdermal patch, a single or multiple number of patches can be used. In a preferred embodiment, the multiple number of patches are used where each patch is identified for use in a given day of treatment. Each patch can contain the same dosing of ghrelin or a variant thereof or the dosing can be tapered as provide previously.

When a single patch is used, the patch can be formulated to provide the same dose per day of ghrelin or a variant thereof. Alternatively, the patch can be formulated so as to provide for a tapering of the dosing of ghrelin or variant thereof over the treatment period.

In one embodiment, this invention provides for a kit of parts comprising a plurality of single dose compositions of ghrelin each composition marked, labeled or otherwise identified for administration on a treatment schedule. In one embodiment, the concentration for each single dose composition of ghrelin is tapered from an initial first dose to a final dose over a period of at least 3 days, preferably at least 5 days and more preferably at least 7 days. In a preferred embodiment, the concentration of ghrelin is reduced from the first dose to the last dose such that the last dose has a ghrelin concentration that is no more than 50% of the first dose.

### EXAMPLE

The following example is illustrative of how this invention can be used.

### Example 1 - Treatment of Sustained mTBl

The drug product is supplied in a 5 mL clear, borosilicate glass vial with a butyl-rubber closure (fluoro-resin film laminated), as a sterile lyophilized white powder or cake equivalent to 14 mg of OXE-103 as the active ingredient and sucrose (inactive ingredient). Matching placebo and diluent product is used.

OXE-103 drug product, placebo, and diluent are stored refrigerated between 2° C. to 8° C. (35.6° F. to 46.4° F.). Reconstituted OXE-103 for SC administration 14 mg (in 5 mL multi-use vials) is stable for up to 14 days at 10° C. or for up to 3 days when stored at 25° C./1000 Lux. The reconstituted drug product (and placebo) is stored refrigerated at 2° C. to 8° C. (35.6° F. to 46.4° F.).

A pilot study to treat sub-acute concussion with ghrelin (OXE-103) is performed. A treatment group (OXE-103) is compared to a placebo group in randomized, double blind fashion and compared using self-report symptom scoring, quality of life questionnaires, computerized cognitive testing assessing executive function, memory and processing speed, and accelerometer-based balance scoring. The exploratory nature of this study is not powered to yield statistically significant outcomes, but allows detection of trends within subjects and between groups, supports comparison with standard tests of neurocognitive functions, and provides sample size estimates for future studies of people with persistent concussion related symptoms.

This study is highly relevant clinically and is the first to test ghrelin as therapy for sub-acute concussion.

### Specific Aims

Describe the change in symptom burden in sub-acute concussion for the two groups. A maximum of 50 subjects are enrolled, but recruiting stops if each arm has 20 participants, diagnosed with persistent concussion symptoms ≤ 28 days post injury who complete the study. Patients are randomized 1:1 to placebo versus OXE-103. The Post-Concussion Symptom Score questionnaire (PCSS) is used as an overall measure of symptom burden. The number of symptoms and severity at each time point are also measures of interest. In addition, subjects are asked to identify and rank the 4 most bothersome symptoms. Without being bound by theory, it is hypothesized that changes in these most bothersome symptoms have a higher correlation to improvement in quality of life. A visual comparison of change in the two groups is made. A change of 20% is considered to be clinically meaningful. This primary aim describes the change in symptoms between day 1 and day 14.

Describe the change in quality of life between the two groups. This aim defines one of the secondary objectives. To assess this, the Quality of Life after Brain Injury scale (QOLIBRI) and a Patient Global Assessment of Status (PGAS) are administered. A change of 20% is considered clinically meaningful. The primary objective with this aim assesses the change between days 1 and 14.

Describe the correlation between the change in symptom burden and quality of life. This is an exploratory aim. Without being bound by theory, it is hypothesized that improvement in symptom burden will correlate with improvement in quality of life measures. This may be more evident in the correlation between the change in the 4 most bothersome symptoms and quality of life measures. This aim describes this correlation between days 1 and 14.

Describe changes between symptom burden and quality of life at different time points. This is an exploratory aim. Data is collected at days 21 and 45 to allow for comparisons at later time points. This may be used to describe changes in these measurements at time points after administration of OXE-103. It is hypothesized that the effects of OXE-103 are long standing and therefore worsening after administration should not occur. Data compare day 14 to days 21 and 45.

Describe changes in cognitive performance between the two groups. This aim defines a secondary objective. Improvement in cognitive functioning could correlate with underlying improvement in neuronal function. Cognitive function is assessed with BrainCheck, a digital assessment tool, at specified intervals. This tool is administered via iPad and can be administered in clinic with supervision by trial personnel as well as at home by the subject.

### Benefits/Risks of Research

Benefits: Currently rest is the initial treatment for concussion. Therapies that can be prescribed later (there is no consensus as to when to start these—tends to range from a couple of weeks post-injury to a couple of months) include physical/vestibular therapy (but this takes time and may provoke symptoms initially), and symptomatic treatment of symptoms with medications. The effectiveness of these drugs to provide potential treatment of underlying neurometabolic changes versus purely symptomatic relief is unknown. Further, each medication comes with potential for adverse events. Providing a safe treatment that is effective at reducing symptoms, increasing quality of life, and potentially providing treatment for underlying neurometabolic dysfunction would be innovative and change the current paradigm of concussion care.

Risks: Previous studies have shown that OXE-103 is quite safe. This study will help to confirm that safety profile in this clinical population. Long term use of ghrelin can lead to increased appetite, weight gain, and adiposity [16]. However, we will mitigate that risk by using OXE-103 for a short period and we will obtain weight measurements at weekly intervals during drug treatment.

### Inclusion/Exclusion

Subjects are both men and women, ages 18-55 years old, with a concussion resulting from a direct or indirect blow, rotation, or whiplash injury to the head or body. They are enrolled within 28 days post injury. Subjects are screened for 7 days to establish stability of their symptoms prior to treatment. Subjects have a symptom severity score of ≥ 20 or higher at the time of randomization (end of screening) in order to reduce the expected degree (number and severity) of spontaneous symptom resolution prior to study completion. Subjects are excluded if during screening they demonstrate 1) improvement of symptom severity or number of symptoms on two consecutive screening assessments or 2) they show a ≥ 20% reduction in symptom severity or number of symptoms.

Subjects with pre-existing neurologic conditions other than mTBI (including cognitive dysfunction) are excluded. Subjects who have been treated with Donepezil (Aricept) and/or memantine (Namenda) after the TBI are excluded.

Subjects receiving other concomitant medications, physical therapy, or other treatments related to their current TBI are eligible 1) if they meet the inclusion criteria related to lack of improvement during the screening period and 2) if such treatments were initiated at least 7 days prior to enrollment and screening. Subjects who are not able to inject themselves are excluded. Ultimately study subject participation is at the discretion of the study physician.

### Study Procedures

### General Study Design

Described herein is a randomized, double-blind, placebo-controlled design where 40 subjects will be randomized to either a placebo cohort or a treatment with OXE-103 cohort. We randomize using the RedCap database. The database is set up to share randomization allocation only with the investigational pharmacy. All subjects are consented and begin the screening period within 28 days post-injury. There is a 7-day screening period prior to randomization and commencement of study treatment to allow for assessment of stability of symptom burden. This screening period must start no later than 28 days post-injury. Starting with Day 1 (end of screening period), the treatment cohort receives OXE-103 40 ug/kg SC twice daily by self-injection and the placebo group receives a placebo injection SC twice daily. Study drug and placebo are maintained and dispensed by Investigational Pharmacy. Subjects receive an 8-day supply of syringes pre-loaded with OXE-103 or placebo. Each cohort receives the 2nd set of syringes with OXE-103 or placebo at the day 7 visit. No therapies other than OXE-103 are administered during the 14-day treatment period to either cohort. After completion of 14 days of either placebo or OXE-103 treatment, starting with the Day 14 visit, other medications and therapies can be initiated for either cohort as needed.

### Subject Training

Subjects are provided with instructions for SC self-administration of OXE-103 and placebo. Subjects are trained to inject themselves and need to demonstrate competency by self-administering the first dose of the study drug at the study site. Alternatively, if a subject is accompanied by a reliable and willing household member, that individual is trained to administer study drug to the subject and will be required to demonstrate competency at the study site. If neither self-administration nor administration by a household member is feasible, the subject will be deemed ineligible to participate. Subjects are instructed on storage of the drug/placebo according to the parameters. A study team member documents the storage location for each subject enrolled. Subjects are asked to inject the first daily dose in the morning, after eating. The second dose occurs in the evening, again after eating.

### Recruitment

Patients meeting the study’s inclusion/exclusion criteria are identified and invited to participate in the study. If they are interested, study team member meets with the patient to discuss the study in more detail. Informed consent is sought; when obtained, subjects are screened for study risks and other exclusion criteria.

Attrition: This is a pilot study and no previous data exists as a basis to estimate attrition for this study. Any study participant who withdraws consent or is removed from the study during the 28-day trial period or does not successfully complete the protocol required 14 days of dosing may be replaced to allow for 40 subjects who complete the protocol.

Target Duration: The target duration of the treatment intervention with OXE-103 is two weeks. The total involvement in the study including screening and follow up assessments are 8 weeks.

### Outcomes & Study Tools

Symptom reduction (AIM 1): Our primary goal is to describe the change in number of symptoms and/or severity in sub-acute concussion with treatment with OXE-103 using the PCSS at days 1 and 15. We will also collect data at days 21 and 44 to potentially describe long term changes and potential lasting effect (AIM 4).

Subjects complete the PCSS at the following timepoints[17]: upon signing consent (score must ≥20) (day -7), mid-way through the screening period (day -3), prior to assignment of a treatment cohort (score must ≥20) (day 1), as well as days 4, 8, 11, 15, 21 and 44. Subjects are instructed to record their symptoms at the same time of day for each assessment timepoint. There can be a two-hour window either way. (e.g. 12PM +/- 2 hrs.) The PCSS is recorded via a RedCap survey. The PCSS is a self-reported assessment of 22 symptoms using a Likert-type scale ranging from 0-6, with 0 indicating no difficulty with the outlined symptom and ratings of 1-6 representing mild-to-severe difficulty with the symptom. The reliability and validity of the PCSS are well documented [18-20] We also ask subjects to rank their 4 most burdensome symptoms (4MBS) at Day 1 and we analyze these separately. As stated in the Specific Aims the purpose of this study is to collect data that would further refine clinical endpoints that could be used for larger Phase 2 studies and possibly lead to the establishment of pivotal endpoints for Phase 3 registration trials. The 22 symptom PCSS assessment is designed to cover the full spectrum of concussion related symptoms across cognitive, emotional and somatic domains. In that regard the PCSS is particularly useful for diagnosis and monitoring recovery of patients post injury. However, due to the number of symptoms across cognitive, emotive and somatic domains, resolution of several mild symptoms may result in a change in the overall symptom score with relatively minor clinical impact on the patient’s well-being. Pre-IND feedback already obtained from the FDA has noted that an effective therapy for concussion must impact the way a patient feels or functions. By including a specific analysis of the patient perceived 4MBS we are likely to be able to correlate symptom scores with improvement in the quality of life tools also being used in the study. The completion of the PCSS will take an estimated 5 minutes.

Quality of Life (AIM 2): A secondary goal is to examine change in quality of life with treatment of sub-acute concussion. Without bewing bound by theory, it is hypothesized that OXE-103 will reduce symptoms when comparing days 1 and 15 and therefore improve quality of life as assessed by 1) Quality of Life after Brain Injury scale (QOLIBRI) and 2) a PGAS.

The QOLIBRI is a 37 item instrument specifically developed to assess health-related quality of life (HRQoL) of individuals after traumatic brain injury [21]. This is built into a RedCap Survey that will be completed online. Since it was developed for TBI as a disease or condition-specific HRQoL instrument, it is expected to be more sensitive than generic quality of life tools. The QOLIBRI was developed by an international task force in two multi-language studies involving over 2000 persons after TBI. Use of a TBI-specific assessment of HRQoL can detect the effects of interventions by measuring physical, psychological, daily life and psychosocial changes typical of TBI. An increase/decrease of 20% in QOLIBRI is judged to represent an improvement.

In addition, a PGAS is used in this study. The tool utilizes a visual analog scale (VAS) to simply assess the patient’s global assessment of their symptoms via the question “How would you rate the effect of your symptoms on how you feel or function today?”. Patients are instructed to use a slider tool within RedCap to rate the effects of their symptoms from 0 to 10 (with 0 being no effect and 10 being worst effect). An increase/decrease of 20% in PGAS is considered to indicate improvement.

The QOL measures are obtained on day 1, 4, 8, 11, 15, 21, & 44. The completion of these takes an estimated 15 minutes.

Cognitive testing (AIM 5): A secondary outcome measure is to summarize change in cognitive function in the 2 groups. Without being bound by theory, it is hypothesized that the mechanism of action of OXE-103 will allow an improvement in cognitive function.

Subjects complete computerized neurocognitive testing on an iPad using BrainCheck [22] BrainCheck is a validated digital assessment tool to aid in the diagnosis of mild cognitive decline. The tool measures a battery of 7 tests to measure cognition, reaction time, and balance. BrainCheck is a Class II medical device by the FDA. BrainCheck assessments include a coordination balance test measuring static and dynamic balance using the Ebbinghaus Illusion, a digit symbol substitution test for general cognitive performance, the Flanker test measuring visual attention, the Stroop Effect measuring reaction time, Trails A&B measuring visual attention and task switching and Recall tests to measure immediate and delayed memory. All scoring algorithms compare test results to a normative age matched dataset. All these tests are simple video games that require no special skills and are expected to cause no distress. The total time to complete the battery of tests is estimated to be 15 minutes. BrainCheck will be conducted in clinic on days 1, 7, 14, and 45; and are conducted by the subject at home on days 3, 10, and 21. The iPads are given to the subjects at the completion of all study procedures as compensation for their time. If a subject withdraws from the study, they will be asked to return the iPad to the study team.

Electronic PHI data is kept on HIPAA compliant servers. The computers are all password protected. Server access is limited to study team and IT representatives. Access to study specific data and communications relating to the study is limited to the PI and PI’s staff, study personnel, responsible individuals from the study sponsor and appropriate regulatory agencies. The research data are be added to the subject’s medical records.

Amazon work servers (AWS) has an established information security organization managed by the AWS security team and is led by the AWS Chief Information Security Officer (CISO).

AWS meets criteria for security, availability, and confidentiality in the American Institute of Certified Public Accountants (AICPA) TSP Section 100, Trust Services Principles and Criteria for security, availability, processing, integrity, confidentiality, and privacy.

BrainCheck uses AWS HIPAA compliant services and holds third-party validations certifying that:
AWS complies with the ISO 27017 implementation guidance of cloud-specific information security controls that supplement the ISO 27002 guidance and the ISO 27001 standard.
AWS complies with the ISO 27001 internationally-recognized standard for security management best practices and comprehensive security controls following the ISO 27002 best practice guidance
AWS complies with the ISO 27018 implementation guidance of controls applicable to public cloud personally identifiable information (PII) protection that supplement the ISO 27002 guidance and the ISO 27001 standard.

Information that will be transmitted from the app is limited to subject code, survey responses, and timestamps of survey responses. No location data will be transmitted. The vendor will not be permitted to attempt to re-identify subjects.

### Statistical Analysis

This pilot study describes changes observed in the two groups: placebo versus OXE-103. Since the study is exploratory, analysis focuses on descriptive comparative statistics and not on a prespecified statistically significant primary endpoint. Data are used to enable power calculations and the definition of suitable clinical endpoints for further clinical development. Data from baseline and on Study Days 1, 4, 8, 11, 15, 21, and 44 will be analyzed.

The primary objective is to determine the proportion of subjects (responders) who experience a clinically meaningful benefit as defined by a reduction of 20% in both the number and severity of concussion related symptoms. Concussion related symptoms are measured using the 22 symptom PCSS. Severity of each of the 22 symptoms is graded by the patient on a 7-point Likert Scale. This scale has been used extensively to assess patients with concussion/mTBI[18, 19].

In addition, in order to control for the effect of changes in clinically minor symptoms on the overall number and severity of the PCSS, data is analyzed based on change of the 4MBS for each subject. This type of analysis has been employed in evaluation of patient reported outcomes for migraine and is discussed in an FDA guidance document (Dodick et al. 2018, Migraine 2018 FDA).

FDA pre-IND guidance from the FDA on the clinical development of OXE-103 advised that “The outcome measure should be constructed in a way that ensures that a score change is indicative of a meaningful improvement in how a patient feels or functions that comes from a treatment effect specific to mild TBI.” In order to correlate changes in symptom number/severity to effect on quality of life, two quality assessment tools are used including QOLIBRI and a PGAS. Improvements in these assessments are compared to definition of Responder to assess meaningful clinical improvements in response to treatment with OXE-103.

In addition, patient reported outcomes on the PCSS scale are correlated with objective digital measures of cognition and balance/stability using BrainCheck a Class-II FDA approved device.

Medical history and test results are housed in a REDCap.

### Plans for Assuring Subjects' Privacy and Confidentiality

Signed consent forms and data forms are stored in a designated file cabinet belonging to a member of the study team with limited access. Outcomes data are entered into the aforementioned REDCap database and access restricted to staff members with approval. All analyzed data are de-identified before submitting to the sponsor or scientific journals, etc. per HIPAA guidelines. Each participant will be assigned a unique study number to allow tracking of information over time. Personally-identifying information is removed from initial data once a study number has been assigned, and from subsequent data once new data has been matched to an existing study number. The identity of participants and their associated study numbers are housed in the Velos system, which will only be accessible by study team members who have authorization to access.

### Follow-Up

Patients will continue with their standard care upon completion of or removal from the study.

Study Design - Schedule of events Cohorts 1 and 2

| Assessment | Screening (Day) | | Day | | | | | | |
|---|---|---|---|---|---|---|---|---|---|
| | -7 (≤ 28 days post injury) | -3 | 1 | 4 | 8 | 11 | 15 | 21 +/- 1 | 44 +/- 3 |
| Clinic Visit | 1 | | 2 | | 3 | | 4 | | 5 |
| Informed Consent | X | | | | | | | | |
| Inclusion/ Exclusion Criteria | X | | | | | | | | |
| Demographics | X | | | | | | | | |
| Body Weight | X | | X | | X | | X | | X |
| Medical History, including current TBI injury, mental health diagnoses, neurologic conditions (including any cognitive dysfunction), and prior treatment with donepezil (Aricept) and memantine (Namenda) | X | | | | | | | | |
| Adverse Effects | | | X | | X | | X | X | X |
| Prior Medications/Therapy Hx | X | | | | | | | | |
| Physical Examination | X | | X | | X | | X | | X |
| Concomitant Medications | X | | X | | X | | X | X | X |
| Train subcutaneous injection * | | | X | | | | | | |
| PCSS | X | X | X | X | X | X | X | X | X |
| Ipad Neurocog and balance (BrainCheck) | | | X | X | X | X | X | X | X |
| QOL - QOLIBRI | | | X | X | X | X | X | X | X |
| QOL- PGAS VAS | | | X | X | X | X | X | X | X |
| Administer OXE-103 40ug/kg SC BID ** | | | X Study Days 1-14 | | | | | | |
| Phone Follow-up | | | | | | | | X | |

* Training on SC injection technique and OXE-103 administration only in Cohort 1
**Assessments and questionnaires performed and collected on Day 7 will be completed prior to administration of OXE-103.

What is claimed is:

1. A method for mitigating one or more symptoms of a mild traumatic brain injury (mTBI) for a patient diagnosed with a sustained mTBI which method comprises administering to the patient an effective amount of ghrelin or a variant thereof over multiple consecutive days after diagnosis.

2. The method of claim 1, wherein ghrelin administration is continued until the patient’s symptoms become asymptomatic.

3. The method of claim 1 or 2, wherein ghrelin or a variant thereof is administered as a pharmaceutical composition.

4. The method of any one of claims 1-3, wherein the pharmaceutical composition is a sterile aqueous solution suitable for injection.

5. The method of claim 4, wherein the pharmaceutical composition is a transdermal patch.

6. The method of any one of claims 1-5, wherein the administration of grhelin or a variant thereof is maintained for a period of at least 3 days after diagnosis of a sustained mTBI.

7. The method of any one of claims 1-6, wherein the administration of grhelin or a variant thereof is maintained for a period of at least 5 days after diagnosis of a sustained mTBI.

8. The method of any one of claims 1-6, wherein the administration of grhelin or a variant thereof is maintained for a period of at least 7 days after diagnosis of a sustained mTBI.

9. The method of any one of claims 1-8, wherein only a single dose of grhelin or a variant thereof is administered per day.

10. The emthod of any one of claims 1-9, wherein the ghrelin or variant is administered by subcutaneous injection.

11. The method of any one of the above claims, wherein ghrelin administration is continued until the patient is able to resume normal activities.

12. A kit of parts comprising a plurality of single dose compositions of ghrelin each composition marked, labeled or otherwise identified for administration on a treatment schedule.

13. The kit of parts of claim 12, wherein each single dose of ghrelin or a variant thereof is tapered from an initial first dose to a final dose.

14. The kit of parts of claim 13, wherein the concentration of ghrelin or a variant thereof is reduced from the first dose to the last dose such that the last dose has a ghrelin concentration that is no more than 50% of the first dose.

15. A method for mitigating one or more symptoms of a mild traumatic brain injury (mTBI) comprising:
a. selecting a patient having mTBI due to an injury and the one or more symptoms of mTBI at least 7 days after the injury;
b. administering ghrelin or a variant thereof to the patient for a period of time, wherein ghrelin or the variant thereof is administered at a dose of about 80 µg/kg per day.

16. The method of claim 15, wherein the ghrelin or variant thereof is administered as about 40 µg/kg twice per day.

17. The method of claim 15 or 16, wherein the period of time is up to about 14 days.

18. The method of claim 17, wherein the period of time is about 14 days.

19. The method of any one of claims 15 to 18, wherein the patient is evaluated for the one or more symptoms of mTBI on scheduled basis.

20. The method of any one of claims 15 to 19, wherein the patient is evaluated for the one or more symptoms of mTBI prior to administration of ghrelin or variant thereof.

21. The method of any one of claims 15 to 20, wherein the patient is evaluated for the one or more symptoms of mTBI during the period of time of administration of ghrelin or variant thereof.

22. The method of any one of claims 15 to 21, wherein the patient is evaluated for the one or more symptoms of mTBI at about 3 days, 7 days, 10 days, 14 days, 20 days, and/or 43 days after initiation of administration of ghrelin or variant thereof.

23. The method of any one of claims 19 to 22, wherein the patient is evaluated using one or more of PCSS, QOLIBRI, PGAS, and/or BrainCheck.
